(19) United States
(12) Patent Application Publication
(10) Pub. No.: US 2026/0123847 A1
(43) Pub. Date: May 7, 2026

(54) FLEXIBLE HYBRID ELECTRONIC SYSTEM FOR INTRAOPERATIVE INTRACRANIAL MONITORING

(71) Applicant: HUAZHONG UNIVERSITY OF SCIENCE AND TECHNOLOGY, Hubei (CN)

(72) Inventors: Hao WU, Hubei (CN); Bo PANG, Hubei (CN); Ganguang YANG, Hubei (CN); Tao HE, Hubei (CN)

(73) Assignee: HUAZHONG UNIVERSITY OF SCIENCE AND TECHNOLOGY, Hubei (CN)

(21) Appl. No.: 19/438,631

(22) Filed: Jan. 1, 2026

Related U.S. Application Data
(63) Continuation of application No. PCT/CN2025/078874, filed on Feb. 24, 2025.

(30) Foreign Application Priority Data
Dec. 31, 2023 (CN) 202311868676.8

(51) Int. Cl. A61B 5/03; A61B 5/00; A61B 5/01; A61B 5/1455; A61B 5/37

(52) U.S. Cl. CPC A61B 5/031; A61B 5/01; A61B 5/14553; A61B 5/37; A61B 5/746; A61B 2562/0247; A61B 2562/0271; A61B 2562/04; A61B 2562/06; A61B 2562/164

(57) ABSTRACT

Disclosed is a flexible hybrid electronic system for intraoperative intracranial monitoring. The system includes a flexible front-end signal collection circuit, a back-end signal processing circuit and a computer system, wherein the front-end signal collection circuit includes a flexible substrate, an electroencephalogram layer, a flexible sensor array, a temperature-controlled hydrogel interface layer, and an electrothermal film. The flexible sensor array is used for collecting pressure, temperature, electroencephalographic and cerebral tissue oxygen saturation signals, and the electrothermal film regulating the adhesion and peeling of the temperature-controlled hydrogel interface layer and cerebral tissue. The signal processing circuit is electrically connected to the signal collection circuit, so as to receive signals collected by the flexible sensor array and the electroencephalographic layer, analyze then transmit to the computer system. When the pressure data exceed a preset normal threshold, the alarm module is triggered to alert the surgeon to the cranial status.

### CROSS-REFERENCE TO RELATED APPLICATION

The present application is a continuation of international PCT application serial No. PCT/CN2025/078874 filed on Feb. 24, 2025, which claims the priority benefit of China application No. 202311868676.8 filed on Dec. 31, 2023. The entirety of the above-mentioned patent application is incorporated herein by reference and made a part of this specification.

### TECHNICAL FIELD

The present disclosure relates to the biomedical devices, and specifically relates to a flexible hybrid electronic system for intraoperative intracranial monitoring.

### DESCRIPTION OF RELATED ART

The human brain, as the advanced central nervous system of the human body, is one of the most critical organs, characterized by its exceedingly complex structure and including a plurality of functional areas. According to data from the International Agency for Research on Cancer, there were 308,102 new cases of central nervous system brain cancer reported in 2020, with 25,139 deaths attributed to brain tumors. The incidence and mortality rates of brain tumors in China rank among the highest globally. Additionally, the mortality rate for patients with severe cranial trauma exceeds 20%, with a severe disability rate of more than 50%. Neurosurgical craniotomy is a crucial method for treating brain tumors and cranial trauma, serving as a necessary intervention when conventional treatments prove ineffective. Given the intricate structure of the cranial brain, which includes numerous central nerves, arterial and venous blood vessels, and functional areas responsible for language and movement, as well as the inherently fragile nature of a cerebral tissue, craniotomy is regarded as one of the most challenging and complex surgical procedures in clinical practice.

Craniotomy primarily involves professional physicians and medical instruments to open the patient's cranial bone, followed by the excision of pathological tissues within the cranial cavity, thereby achieving therapeutic objectives. Craniotomy may be classified into bone window craniotomy and bone flap craniotomy, and it is utilized for treating diseases related to the cranium and brain. Craniotomy is a common neurosurgical procedure. The success rate of craniotomy varies depending on the specific disease being treated. Data indicate that the success rate of craniotomy for the removal of brain tumors is approximately 90%, while the success rate for craniotomy addressing cerebrovascular diseases generally ranges from 90% to 95%. However, the success rate may be lower for craniotomy performed on functional system diseases. One of the most common reasons for failure during the intracranial surgical phase is brain contusion and hemorrhage caused by the traction of the cerebral tissue. To provide the operator with sufficient visibility, it is necessary to retract the intracranial cerebral tissue during craniotomy. If the cerebral tissue is retracted improperly or for an inappropriate duration during the surgery, it could lead to cerebral tissue hemorrhage, resulting in brain contusion and hemorrhage. In severe cases, complications such as hypothalamic dysfunction may occur. Given the fragile nature of the cerebral tissue, utmost care should be taken to protect the cerebral tissue during craniotomy. Therefore, when necessary retraction is performed, cottonoid patties should be placed at the retraction site.

The existing cottonoid patties are primarily composed of medical degreased patties or spunlace non-woven fabric. Serving as a thin and soft cushioning layer, these patties mitigate the pressure exerted by surgical instruments on the surrounding intracranial tissues during surgical procedures, thus safeguarding the neural tissues within the surgical area. This protection significantly reduces the risks of postoperative hemorrhage, cerebral contusions, and other complications. Direct contact between surgical instruments and cerebral tissues presents a substantial mechanical disparity between the conventional materials used in brain retractors and brain electrodes and the cerebral tissues themselves, which may easily lead to tissue damage. Improper handling during such operations considerably elevates the risk of postoperative complications and increases the likelihood of fatal or disabling outcomes.

Due to the difficulty and complexity of neurosurgical craniotomy procedures, it is imperative for surgeons to constantly ensure during the operation that the surgical steps being undertaken do not cause damage to the patient's nerves, to ascertain whether the patient's vital signs are stable, and to ensure the accuracy of critical cerebral parameters to prevent postoperative complications. In addition to utilizing microscopic observation, intraoperative neurophysiological monitoring (IONM) may detect information imperceptible to the naked eye, thus reflecting various physiological indicators of the human body. Consequently, during surgery, it is essential to monitor and promptly feedback data on intracranial physiological parameters, including cerebral tissue oxygen saturation, brain electrical signals, temperature, and the pressure exerted by surgical instruments on surrounding tissues. Conventional brain patties typically do not possess transparency or the capability to provide monitoring feedback, and the limited space available during craniotomy makes it challenging to conduct physiological monitoring while simultaneously protecting the brain. This limitation hinders the continuous implementation of electrophysiological monitoring. Ordinarily, the surgeon operates during brain protection and conducts electrophysiological monitoring at intervals, which makes it difficult to ensure comprehensive monitoring of the patient's brain condition throughout the several-hour-long surgery.

Cerebral tissue oxygen saturation typically reflects the balance between cerebral oxygen supply and demand. Given the high metabolic rate of cerebral tissue, it is particularly sensitive to hypoxic conditions, where even brief periods of hypoxia may cause irreversible damage to the central nervous system. Monitoring cerebral oxygen saturation is primarily used to assess the cerebral oxygen balance in patients. Intraoperative monitoring of cerebral oxygen saturation aids in the timely detection of cerebral hypoxia and ischemia. This is of crucial importance in major neurosurgical procedures, large vessel surgeries, emergency and critical care patient management, and cerebral protection during cardiopulmonary resuscitation following cardiac arrest. Anesthesia during surgery may easily disrupt this cerebral oxygen balance, increasing the incidence of postoperative neurological dysfunction. Electroencephalographic (EEG) monitoring is mainly used to determine the presence of brain waves in patients. Intracranial EEG, free from interference by the scalp and skull, may be placed in deeper brain regions, providing surgeons with more detailed electroencephalographic information.

In recent years, the application of flexible electronics in cerebral physiological monitoring has become increasingly widespread. It is used in neurosurgery for intraoperative recording, postoperative rehabilitation monitoring, and neural stimulation tasks. Compared to conventional silicon-based electronics, flexible electronics offer advantages such as excellent ductility, stretchability, and biocompatibility, partially addressing the issue of continuous monitoring. These devices may conformally contact cerebral tissue, effectively reducing damage to the brain. In neurosurgical procedures, damage to cerebral tissue primarily arises from the traction, displacement, and bending caused by surgical instruments, all of which translate into pressure on the cerebral tissue. Therefore, the development of devices capable of measuring cerebral pressure is of significant necessity.

### SUMMARY

To address the issues present in the background technology, the present disclosure provides a flexible hybrid electronic system for intraoperative intracranial monitoring. This flexible hybrid electronic system may serve as a substitute for conventional cottonoid patties, functioning to retract cerebral tissue and expose the lesion area. Additionally, the flexible hybrid electronic system is capable of continuously monitoring the state of cerebral tissue, thereby ensuring safety during surgery.

The technical solution of the present disclosure for solving the above technical problems is as follows:

The present disclosure provides a flexible hybrid electronic system for intraoperative intracranial monitoring, including a flexible front-end signal collection circuit, a back-end signal processing circuit, and a host computer system.

The flexible front-end signal collection circuit includes a flexible framework body formed by a top encapsulation layer, an intermediate encapsulation layer, and a bottom substrate layer arranged from top to bottom, an electroencephalographic layer arranged between the intermediate encapsulation layer and the top encapsulation layer, a flexible sensor array disposed on an upper surface of the bottom substrate layer, a temperature-controlled hydrogel interface layer integrated above the top encapsulation layer, and an electrothermal film integrated below the bottom substrate layer. The electroencephalographic layer includes a top substrate layer and an electroencephalographic electrode array, and an internal circuit disposed on the top substrate layer. The electroencephalographic electrode array passes through the flexible framework body and connects with the temperature-controlled hydrogel interface layer. The flexible sensor array connects with the electroencephalographic layer through the intermediate encapsulation layer. The flexible sensor array is provided to detect a pressure signal, a temperature signal, and a cerebral tissue oxygen saturation signal. The electrothermal film controls the adhesion of the temperature-controlled hydrogel interface layer through temperature, achieving the adhesion and peeling of the temperature-controlled hydrogel interface layer with cerebral tissue.

The back-end signal processing circuit is electrically connected with the flexible front-end signal collection circuit to receive the pressure signal, the temperature signal, an electroencephalographic signal, and the cerebral tissue oxygen saturation signal, analyze and process the signals, and then transmit the signals to the host computer system through a wireless or wired transmission method.

According to the above solution, the flexible sensor array includes a pressure sensor array, a temperature sensor array, and a cerebral tissue oxygen sensor, which are respectively electrically connected with the back-end signal processing circuit.

According to the above solution, a plurality of pressure sensors in the pressure sensor array are all flexible pressure sensors.

According to the above solution, the back-end signal processing circuit includes a collection circuit, a core processing circuit, a multiplexer switch, a flexible battery, a power management chip, and a wireless transmitter. The flexible battery provides a power supply to the wireless flexible hybrid electronic system through the power management chip. The collection circuit is respectively electrically connected with the flexible sensor array and the electroencephalographic electrode array, configured to receive the pressure signal, the temperature signal, the electroencephalographic signal, and the cerebral tissue oxygen saturation signal, and transmit the signals to the core processing circuit through the multiplexer switch. The core processing circuit is configured to process the signals transmitted by the collection circuit and send the signals to the host computer system.

According to the above solution, the collection circuit includes an operational amplifier, an electroencephalogram collection front-end, and a cerebral oxygen collection front-end. The operational amplifier is electrically connected with the pressure sensor array and the temperature sensor array. The electroencephalogram collection front-end is electrically connected with the electroencephalographic electrode array. The cerebral oxygen collection front-end is electrically connected with the cerebral tissue oxygen sensor.

According to the above solution, the core processing circuit transmits the processed signals to the host computer system through the wireless transmitter or through a serial port.

According to the above solution, the materials of the top encapsulation layer, the intermediate encapsulation layer, the bottom substrate layer, and the top substrate layer in the flexible front-end signal collection circuit are all polydimethylsiloxane. The electroencephalographic electrode array, the internal circuit, and the wires connecting various components of the wireless flexible hybrid electronic system are all prepared from flexible composite conductive materials.

According to the above solution, the temperature-controlled hydrogel interface layer includes a plurality of temperature-controlled hydrogel patches corresponding to electroencephalographic electrodes one by one in the electroencephalographic electrode array. The plurality of temperature-controlled hydrogel patches respectively cover the corresponding electroencephalographic electrodes. The temperature-controlled hydrogel patches are formed through photo-initiated polymerization using poly (ethylene glycol) diacrylate as an initiator and poly(3,4-ethylenedioxythiophene) and poly (styrene sulfonate) as blending components.

According to the above solution, a thermally-induced actuator is disposed in the electrothermal film. Heating sites of the thermally-induced actuator correspond to positions of the temperature-controlled hydrogel patches one by one.

According to the above solution, the flexible front-end signal collection circuit and the back-end signal processing circuit are connected through the flexible printed circuit (FPC).

The host computer system is provided with an alarm module. When the pressure data received in the host computer system is higher than a preset normal pressure, the alarm module activates the alarm.

The advantageous effects of the present disclosure are as follows: The flexible hybrid electronic system of the present disclosure may serve as a substitute for conventional brain patties, functioning to tug cerebral tissue and expose the lesion area. Additionally, the flexible hybrid electronic system of the present disclosure is capable of monitoring electroencephalography data, pressure data, temperature data, and cerebral tissue oxygen saturation level data throughout the process. Furthermore, the flexible hybrid electronic system of the present disclosure may provide alerts when necessary, thereby aiding in the improvement of the success rate of craniotomy surgeries.

### BRIEF DESCRIPTION OF THE DRAWINGS

FIG. 1 is a structural schematic diagram of a flexible hybrid electronic system for intraoperative intracranial monitoring of the present disclosure.

FIG. 2 is a fabrication process diagram of an electroencephalographic layer and a bottom substrate layer of the flexible hybrid electronic system for intraoperative intracranial monitoring of the present disclosure.

FIG. 3 is a system integration process diagram of the flexible hybrid electronic system for intraoperative intracranial monitoring of the present disclosure.

FIG. 4 is a circuit schematic diagram of the flexible hybrid electronic system for intraoperative intracranial monitoring of the present disclosure.

FIG. 5 is a host computer software of the flexible hybrid electronic system for intraoperative intracranial monitoring of the present disclosure.

FIG. 6 is a data playback interface of the flexible hybrid electronic system for intraoperative intracranial monitoring of the present disclosure.

FIG. 7 illustrates a method diagram of use of the flexible hybrid electronic system for intraoperative intracranial monitoring of the present disclosure.

### DESCRIPTION OF THE EMBODIMENTS

The principles and features of the present disclosure are described below in conjunction with the drawings and specific embodiments. The examples are only used to explain the present disclosure and are not intended to limit the scope of the present disclosure.

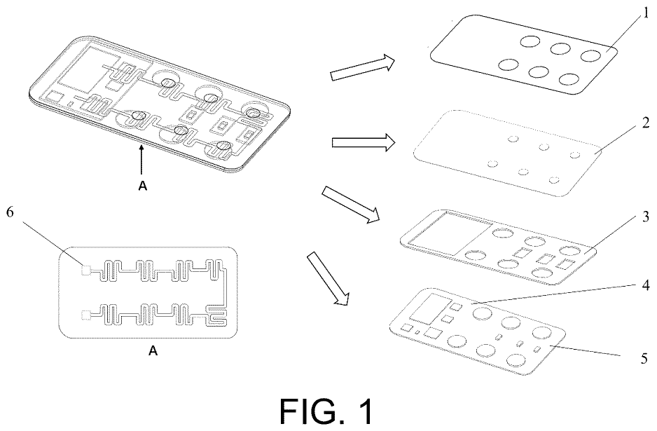

The present disclosure provides a flexible hybrid electronic system for intraoperative intracranial monitoring. The structure is shown in FIG. 1, including a flexible front-end signal collection circuit 31 and a back-end signal processing circuit 4. The flexible front-end signal collection circuit 31 includes a flexible framework body formed by a top encapsulation layer 1, an intermediate encapsulation layer 3 and a bottom substrate layer 5-1 arranged from top to bottom, an electroencephalographic layer 2 disposed between the intermediate encapsulation layer 3 and the top encapsulation layer 1, a flexible sensor array 5 disposed on an upper surface of the bottom substrate layer 5-1, a temperature-controlled hydrogel interface layer 10 integrated above the top encapsulation layer 1, and an electrothermal film 6 integrated below the bottom substrate layer 5-1. The electroencephalographic layer 2 includes a top substrate layer 2-1 and an electroencephalographic electrode array 24 and an internal circuit disposed on the top substrate layer 2-1. The electroencephalographic electrode array 24 extends beyond the flexible framework body through a window on the top encapsulation layer 1 and is connected with the temperature-controlled hydrogel interface layer 10. The flexible sensor array 5 passes through a window of the intermediate encapsulation layer 3 to connect with the electroencephalographic layer 2. The flexible sensor array 5 is provided to detect a pressure signal, a temperature signal, and a cerebral tissue oxygen saturation signal. The electrothermal film 6 adjusts the adhesion of the temperature-controlled hydrogel interface layer 10 through temperature adjustment to achieve adhesion and peeling between the temperature-controlled hydrogel interface layer 10 and a cerebral tissue. The back-end signal processing circuit 4 is electrically connected with the flexible front-end signal collection circuit 31 to receive the pressure signal, the temperature signal, an electroencephalographic signal, and the cerebral tissue oxygen saturation signal, analyze and process the signals, and then transmits the signals to the host computer system through wireless or wired transmission.

According to the above solution, the flexible sensor array 5 includes a pressure sensor array 21, a temperature sensor array 22 and a cerebral tissue oxygen sensor, which are respectively electrically connected with the back-end signal processing circuit 44.

The electroencephalographic electrode array 24 includes a plurality of electroencephalographic electrodes. The plurality of electroencephalographic electrodes are electrically connected through the internal circuit, and are electrically connected with the back-end signal processing circuit 44 through the internal circuit.

According to the above solution, a plurality of pressure sensors 7 in the pressure sensor array 21 are all flexible pressure sensors. The pressure sensor 7 adopts medical patties as the backbone and uses polymer material PEDOT.PSS as a conductive channel of a piezoresistive material to obtain the high-performance piezoresistive pressure sensor 7. In addition to having good flexibility and biocompatibility, the pressure sensor 7 also has a high-pressure range and a high response speed. Signal acquisition and transmission between the plurality of pressure sensors 7 is conducted using wires made from flexible composite conductive materials.

In some embodiments, the temperature sensor array 22 includes a plurality of temperature sensors 9, which are thermistor-based temperature sensors. These thermistors may be either negative temperature coefficient thermistors (NTC) or positive temperature coefficient thermistors (PTC), where the resistance value of the thermistor varies with temperature changes. For the temperature sensors 9 and the interfaces, a standard platinum resistance thermometer is used in a constant temperature bath to conduct a heating comparison, verifying the accuracy of the temperature under heating conditions and the phase transition characteristics of the interface.

Thermistors exhibit high sensitivity, small size, ease of use, and good stability. For the temperature sensors 9 and interfaces, a standard platinum resistor is utilized in a constant temperature water bath for heating comparison to verify the accuracy of temperature under heating conditions and the phase transition characteristics of the interface. The resistance-temperature characteristics of a negative temperature coefficient (NTC) thermistor may be approximately represented by the formula

$${R}_{t} = R \times {e}^{{B}_{n} ( \frac{1}{{T}_{1}} - \frac{1}{{T}_{2}} )} .$$

The resistance-temperature characteristics of a positive temperature coefficient (PTC) thermistor within a working temperature range may be approximately represented by the formula Rₜ=R×e^{B}^{ₚ}^{(t}^{₁}^{-T}^{₂₎}, wherein T₁ and T₂ are Kelvin temperatures, Rₜ is a resistance of the thermistor at temperature T₁, R is a nominal resistance of the thermistor at normal temperature T₂, which generally refers to an actual resistance of the thermistor when the ambient temperature is 25 degrees Celsius, Bₚ and Bₙ are material constants of the thermistor, that is, a sensitivity index, the greater the Bₚ and Bₙ values, the higher the sensitivity of the thermistor. In fact, the Bₚ and Bₙ values of the thermistor are not constant, and a magnitude of their changes varies depending on the material composition.

In some specific embodiments, the cerebral tissue oxygen sensor includes a photoelectric converter and a light source. The light source adopts two light-emitting diodes (LED) 27 with selectively different specific wavelengths for oxyhemoglobin and deoxyhemoglobin. The device employs a photodiode 26 to receive light reflected and absorbed by cerebral tissue, converting the light signal into an amplified output. Ultimately, data processing is conducted according to the Lambert-Beer law to derive the cerebral tissue oxygen saturation at the respective moment. It is required that the incident light source and receiving light source remain unobstructed or made from transparent materials.

In some specific embodiments, the back-end signal processing circuit 4 converts a cerebral tissue oxygen near-infrared spectrum of red light and infrared light collected by the flexible front-end signal collection circuit 31 into cerebral oxygen saturation output through a specific cerebral oxygen saturation algorithm. The process of the specific cerebral tissue oxygen algorithm is as follows.

Step 1: A sampling rate of a data collection front-end detection circuit is set to 150 Hz, with a sampling duration of 2 seconds. The acquisition process commences when a detection circuit of the back-end circuit detects power-on, subsequently controlling the light-emitting diode 27 to emit red light and infrared light. The optical signals are received by the photodiode 26 on a receiving end. Once the data reaches a specific duration, processing begins by applying a low-pass filter at 0.5 Hz to the waveform. After passing through the data collection front-end, the direct current (DC) components of the red light and infrared light are obtained. Each DC component is then subjected to a band-pass filter ranging from 0.5 Hz to 5 Hz, thereby yielding the requisite raw data.

Step 2: The Lambert-Beer law formula OD(λ, t)=−ln(Iₜ(λ)/I₀(λ)) is applied to determine the optical densities for red light and infrared light separately. Subsequently, the amounts of red light and infrared light absorbed by hemoglobin, denoted as Hb(t) and HbO₂(t) respectively, are calculated.

Step 3: The tissue oxygen saturation is obtained by calculating a ratio of concentrations of hemoglobin and oxyhemoglobin in the blood. The formula is as follows:

$$rSt {O}_{2} ( t ) = \frac{Hb {O}_{2} ( t )}{Hb {O}_{2} ( t ) + Hb ( t )} .$$

Here, rStO₂ represents a value of tissue oxygen saturation.

Step 4: The calculated signal of tissue oxygen saturation is transmitted to a mobile terminal App 12 and the host computer software 13 via a Bluetooth module. When the back-end signal processing circuit 4 receives data reaching a length of 0.5 seconds again, a window with a time domain length of 2 seconds will move backward by a data length of 0.5 seconds and repeat the aforementioned steps to calculate the cerebral oxygen saturation signal.

The preparation of the flexible front-end signal collection circuit 31 combines flexible electronic manufacturing process and micro-nano processing process, and the materials of each layer are connected by oxygen plasma bonding.

According to the above solution, the materials of the top encapsulation layer 1, the intermediate encapsulation layer 3, the bottom substrate layer 5-1, and the top substrate layer 2-1 in the flexible front-end signal collection circuit 31 are all polydimethylsiloxane (PDMS). A mass ratio of a prepolymer and a curing agent of PDMS is 10:1, which are added into a plastic cup in sequence and stirred continuously for 5 minutes to ensure that the curing agent and prepolymer are mixed evenly, so as to prepare the top encapsulation layer 1, the intermediate encapsulation layer 3, the bottom substrate layer 5-1, and the top substrate layer 2-1.

The electroencephalographic electrode array 24, the internal circuit, and the wires connecting the components of the wireless flexible hybrid electronic system are all made of flexible composite conductive materials.

In some specific embodiments, masks are fabricated on the bottom substrate layer 5-1 and the top substrate layer 2-1 by laser cutting, and the flexible electrically conductive composites (ECC) is coated on the printed circuit board by scraper coating, and then placed on an adjustable temperature hot plate with a temperature set to 160 degrees Celsius and a heating time of 30 minutes. The ECC is composed of silver flakes dispersed in PDMS, which serves as a material for electrodes and interconnects, enabling the overall system to be mechanically stretchable and highly conductive. The flexible electrically conductive composites (ECC) obtained by mixing PDMS and silver powder according to a mass ratio of 1:3.

When preparing the electroencephalographic electrodes using the flexible composite conductive material, in order to ensure that the material has reliable biocompatibility, gold is sputtered on the surface of the electroencephalographic electrodes as a protective layer and an adhesion layer for the interface material.

According to the above solution, the temperature-controlled hydrogel interface layer 10 includes a plurality of temperature-controlled hydrogel patches corresponding to the electroencephalographic electrodes one by one, and the plurality of temperature-controlled hydrogel patches respectively cover the corresponding electroencephalographic electrodes. The temperature-controlled hydrogel patches are formed by photo-initiated polymerization using poly(ethylene glycol) diacrylate as an initiator and poly(3,4-ethylenedioxythiophene) and poly(styrene sulfonate) as blending components.

The acquisition of electroencephalographic signals often occurs intraoperatively for the measurement of neuro-evoked potentials. Therefore, during surgery, the interface must exhibit high adhesion, securely adhering to the brain, effectively reducing the generation of stimulation artifacts. When repositioning is necessary or upon the conclusion of the surgery when it is required to move the brain protection device, the electrothermal film 6 is activated, raising the temperature above a phase transition point. Upon dehydration, hydrogel microspheres rapidly lose adhesion, allowing the system to be removed without adhering to the cerebral tissue. A phase transition temperature of the interface is lower than a brain temperature.

Specifically, gold sputtered on the electroencephalographic electrodes facilitates the grafting of the interface, and the hydrogel is cast on the electroencephalographic electrodes. When the temperature is low, a surface of the temperature-controlled hydrogel patch has adhesion and conformally adheres to the cerebral tissue to achieve stable transmission of the electroencephalographic signals. After the surgery is completed, the electrothermal film 6 is activated, and the heating causes the temperature to rise, the interface is de-adhered, and the flexible hybrid electronic system is removed without the temperature-controlled hydrogel patch adhering to the cerebral tissue.

According to the above solution, a thermally-induced actuator 30 is provided in the electrothermal film 6, and the thermally-induced actuator 30 is powered by a flexible battery 28.

The heating sites of the thermally-induced actuator 30 correspond to the positions of the temperature-controlled hydrogel patches one by one. By altering the temperature through the thermally-induced actuator 30, the temperature-controlled hydrogel patches may be made to adhere or detach.

In some specific embodiments, the thermally-induced actuator 30 is a resistance wire, and the resistance wire is laid flat in the electrothermal film in a meandering manner. When the practitioner needs to change the position or move the brain protection device after the surgery is completed, the electrothermal film 6 is activated to set the temperature to be greater than the phase transition temperature, so that the microspheres in the temperature-controlled hydrogel interface are rapidly de-adhered after dehydration, and the system may be removed without adhering to the cerebral tissue. An infrared camera is used to capture the heating process of the heater to test the uniformity of heating and the temperature rise curve characteristics of the system.

An alarm module is provided in the host computer system. When the pressure data received in the host computer system is higher than a preset normal pressure, the alarm module activates an alarm.

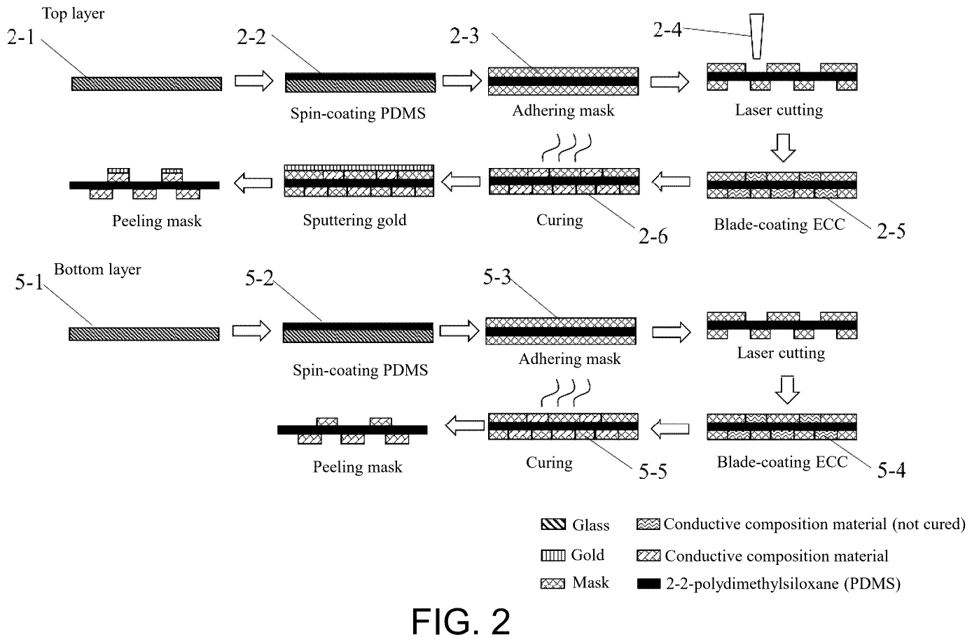

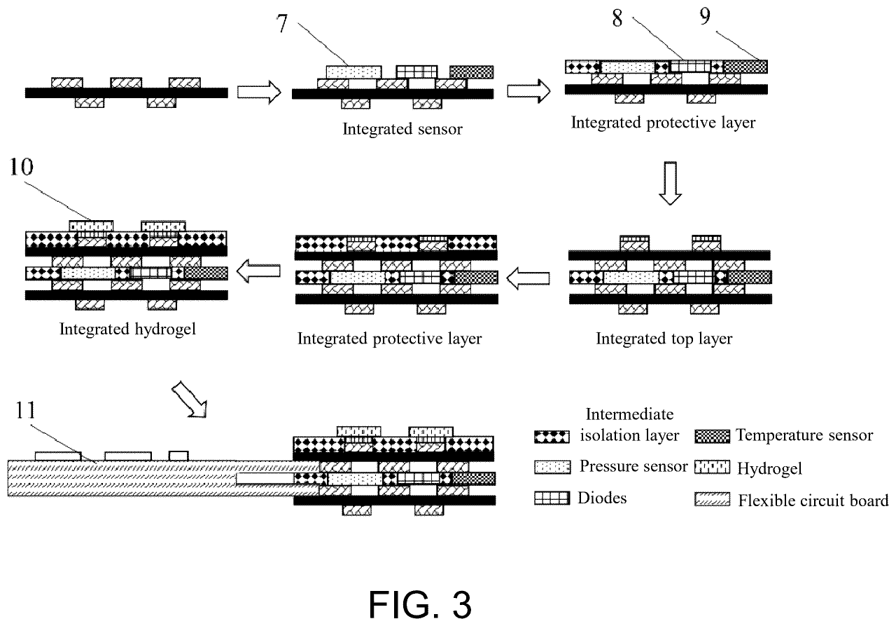

The preparation process of the flexible hybrid electronic system for intraoperative intracranial monitoring of the present disclosure is shown in FIG. 2 and FIG. 3.

### (1) Preparation of the Electroencephalographic Layer 2

S1: The PDMS 2-2 is spin-coated on a glass plate 2-1 to form the top substrate layer, wherein the mass ratio of the prepolymer and the curing agent of the PDMS is 10:1. After mixing uniformly, curing is performed in an oven at a temperature of 90 degrees Celsius for a heating time of 30 minutes. After curing is completed, the PDMS layer is peeled off from the glass plate, and mask materials 2-3 are adhered to both sides of the PDMS layer.

S2: A laser cutter 2-4 is utilized to perform laser cutting on the mask to cut out the required electroencephalographic electrode patterns and internal circuit patterns. After cutting is completed, conductive composite material (ECC) 2-5 is blade-coated on the mask, wherein the ECC is composed by mixing PDMS and silver powder in a mass ratio of 1:3. After blade-coating is completed, a cured conductive composite material 2-6 is formed.

S3: Gold is sputtered on the cured electroencephalographic electrodes, and finally the mask is peeled off from the substrate layer to obtain the electroencephalographic layer 2.

### (2) Preparation of the Bottom Substrate Layer 5-1

S1: PDMS 5-2 is spin-coated on a glass plate 5-1 to form the bottom substrate layer, wherein the mass ratio of the prepolymer and the curing agent of PDMS is 10:1. After mixing uniformly, curing is performed in an oven at a temperature of 90 degrees Celsius for a heating time of 30 minutes. After curing is completed, the PDMS layer is peeled off from the glass plate, and masks 5-3 are adhered to both sides of the PDMS layer.

S2: A laser cutter is utilized to perform laser cutting on the mask 5-3 to cut out the required conductive layer patterns. After cutting is completed, electrically conductive composites (ECC) 5-4 is blade-coated on the mask, wherein the ECC is composed by mixing PDMS and silver powder in a mass ratio of 1:3. After blade-coating is completed, a cured conductive composite material 5-5 is formed.

S3: Finally, the mask is peeled off from the substrate layer to obtain the bottom substrate layer 5-1 with a conductive layer.

### (3) Integration of the Flexible Hybrid Electronic System

S1: A plurality of pressure sensors 7, temperature sensors 9, and diodes 8 (including photodiodes 26 and light-emitting diodes 27) for measuring cerebral tissue oxygen are integrated on the above-prepared bottom substrate layer 5-1 with the conductive layer. After integration, the intermediate encapsulation layer 3 is integrated, wherein the intermediate encapsulation layer 3 has windows through which the pressure sensors 7, the temperature sensors 9, the photodiodes 26 and the light-emitting diodes 27 reach out, thus providing protection for the flexible sensor array 5.

S2: The electroencephalographic layer 2 and the top encapsulation layer 1 are sequentially integrated on an upper end of the intermediate encapsulation layer 3.

S3: The temperature-controlled hydrogel interface layer 10 is integrated above the top encapsulation layer 1, wherein the layers are bonded through plasma bonding. After all bonding is completed, the flexible front-end signal collection circuit 31 of the system is obtained.

S4: The flexible front-end signal collection circuit 31 is connected with the back-end signal processing circuit 4.

According to the above solution, the back-end signal processing circuit 4 includes a hardware collection circuit board, the flexible battery 28, a power management chip 29, a core processing circuit 18, a multiplexer switch 19 and a wireless transmitter disposed on the hardware collection circuit board. The flexible battery 28 achieves a power supply for the wireless flexible hybrid electronic system through the power management chip 29. The collection circuit is electrically connected with the flexible sensor array 5 and the electroencephalographic electrode array 24, respectively, for receiving the pressure signal, the temperature signal, the electroencephalographic signal, and the cerebral tissue oxygen saturation signal and transmitting the signals to the core processing circuit 18 through the multiplexer switch 19. The core processing circuit 18 is configured for processing the signals transmitted by the collection circuit 4 and sending the signal to the host computer system, wherein the hardware collection circuit board adopts a flexible circuit board FPC 11.

According to the above solution, the collection circuit includes an operational amplifier 20, an electroencephalogram collection front-end 23, and a cerebral oxygen collection front-end 25. The operational amplifier 20 is electrically connected with the pressure sensor array 21 and the temperature sensor array 22. The electroencephalogram collection front-end 23 is electrically connected with the electroencephalographic electrode array 24. The cerebral oxygen collection front-end 25 is electrically connected with the cerebral tissue oxygen sensor.

The microcontroller unit (MCU) master control chip of the core processing circuit 18 is a CY8CKIT-062-BLE chip, which internally integrates a clock management circuit, a Bluetooth SoC, multiple ADCs, and interfaces such as SPI and I2C. A clock oscillator includes a 32.768 kHz timer clock circuit and a 32 kHz high-speed clock. The MCU master control chip is directly connected to the signal input end of a Bluetooth antenna in a Bluetooth module, transmitting signals externally through the antenna, and employs a symmetric encryption protocol to ensure the security of data transmission. The MCU master control chip may also use a UART serial port 17 to send raw data to the host computer software 13. In an embodiment, the program on the computer may be pre-burned into the chip before integrating the chip into the circuit.

According to the above solution, the core processing circuit 18 transmits the processed signals to the host computer system through the wireless transmitter or through the serial port 17.

In some specific embodiments, the flexible circuit board FPC 11 and the front-end interface of the collection circuit are connected through snap-fit or anisotropic conductive film (ACF) connection.

In some embodiments, the hardware collection circuit board of the back-end signal processing circuit 4 employs a modular design, including a plurality of collection board cards that allow for modular combinations of collected parameters. These board cards are categorized into power management areas, main control areas, data collection areas, signal storage areas, and communication areas. The data acquired by the rear end of the collection circuit is transmitted to the hardware board cards, which, through their modular design, send the raw data to the host computer system via Bluetooth, Wi-Fi, or serial port for analysis, providing a real-time display of the current state of cerebral tissue and offering safety monitoring for the operating physician and anesthesiologist. The front end of the collection circuit typically deals with analog signals, utilizing a voltage divider resistor principle and analog-to-digital conversion (ADC) to acquire pressure and temperature signals, with a load capacity enhanced by a follower operational amplifier 20. The signals from the electroencephalogram electrode array 24 are collected using a specialized chip (ADS1299), which performs multi-channel data collection at a sampling rate of 16k, and these signals are transmitted to the host computer system via wireless communication and serial transmission. The power management chip 29 employs DC-DC and LDO methods to ensure the stability of data collection and transmission.

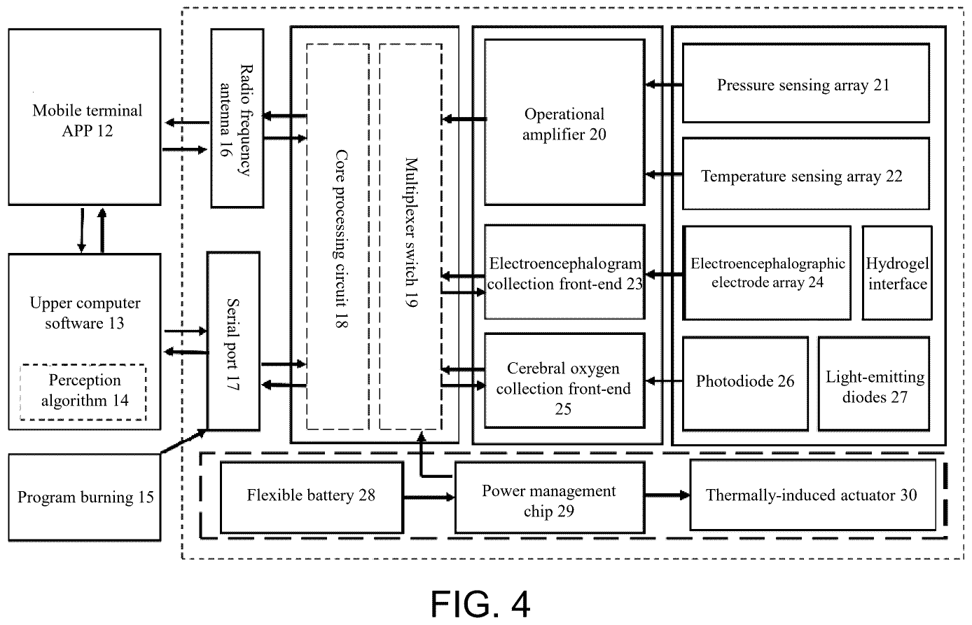

As depicted in FIG. 4, the schematic diagram illustrates a wireless flexible hybrid electronic system for intraoperative intracranial monitoring. Initially, the pressure sensor array 21 and the temperature sensor array 22 collect pressure and temperature signals, which are then processed by the operational amplifier 20. The operational amplifier 20 serves to follow and enhance the load-driving capability of the signal. The electroencephalographic signals and cerebral tissue oxygen saturation signals captured by the electroencephalographic electrode array 24, the photodiode 26, and the light-emitting diode 27 are transmitted to the electroencephalographic collection front-end 23 and the cerebral oxygen collection front-end 25, respectively. Subsequently, the signals obtained by the operational amplifier 20, the electroencephalographic collection front-end 23, and the cerebral oxygen collection front-end 25 are transmitted through the multiplexer switch 19 to the core processing circuit 18. The multiplexer switch 19 is capable of outputting the input the electroencephalographic signal, the cerebral oxygen saturation signal, the temperature signal, and the pressure signal onto a single output line. The core processing circuit 18 then performs digital filtering, 50 Hz notch filtering, and wavelet transformation on the existing data. The processed signals may be transmitted to a mobile terminal APP 12 via Bluetooth, NFC, or WIFI radio frequency antenna 16. Furthermore, the core processing circuit 18 may transmit multimodal data to the host computer software 13 through the serial port 17, where algorithm processing is performed using MATLAB. A state perception algorithm 14 is embedded within the host computer software 13 to display various data from the flexible front-end signal collection circuit 31 and the current operational state of the cerebral tissue. It shows the waveform of the measured data and the current safety status, ensuring the safe conduct of neurosurgical operations.

In some specific embodiments, the firmware can be burned onto the chip from a computer via a dedicated programmer, after which the chip is integrated into the circuit. Finally, the collection circuit, the core processing circuit 18 and the thermally-induced actuator 30 are powered by the flexible battery 28 through the power management chip 29. The power management chip 29 includes multiple DC-DC and LDO, ensuring stable transmission of data.

The host computer system may be the mobile terminal APP 12 or the host computer software 13. The host computer software 13 is designed to adopt a visual interface form for display, after the multimodal data is transmitted to the host computer system, MATLAB is first used for algorithm processing. The state perception algorithm is embedded in the host computer display software, used for identifying and warning the potential harm to the cerebral tissue caused by improper operations during surgery. The host computer system may display various data of the flexible front-end signal collection circuit 31 and the cerebral tissue status of the current operation process, ensuring the safe performance of neurosurgery. After performing digital filtering, 50 Hz notch filtering and wavelet transform on the existing data, combined with the optical processing method of Lambert-Beer law, the current intraoperative cerebral tissue status is determined in real-time, and transmitted to the host computer visual interface written in Python, displaying the measured data waveforms and the current safety status.

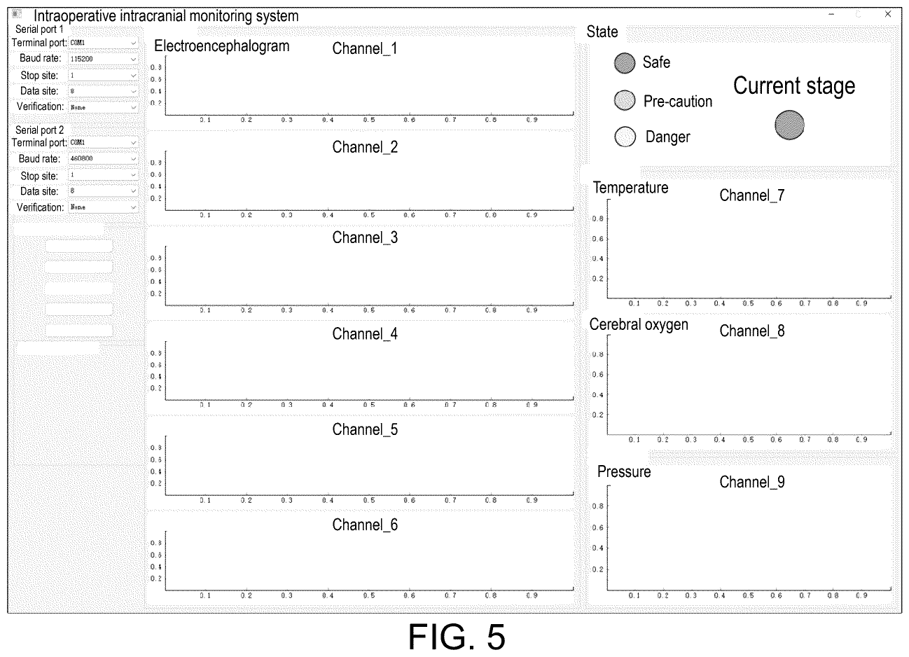

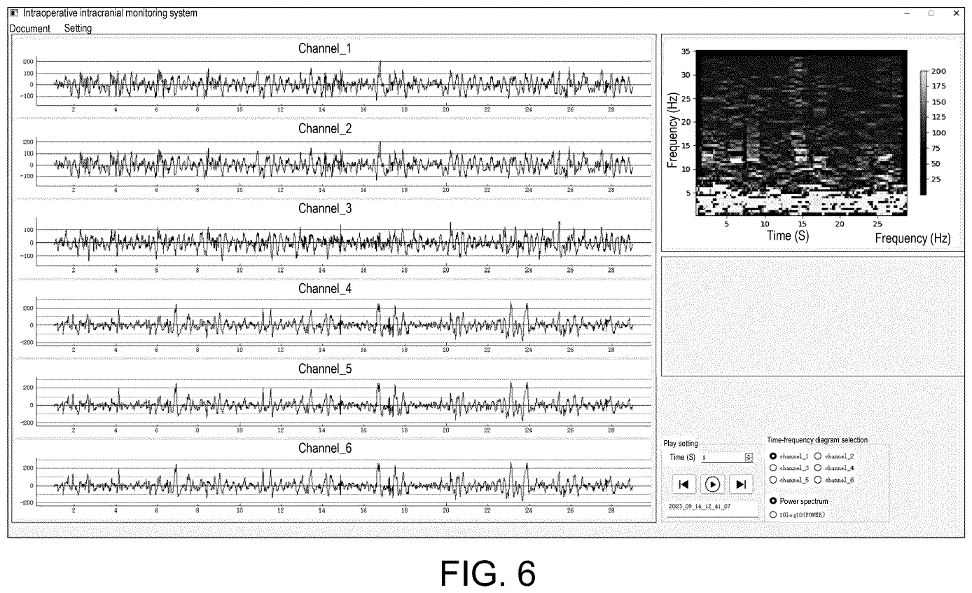

As depicted in FIG. 5 and FIG. 6, a user interface of the host computer software 13 is presented. FIG. 5 illustrates a software interface in the absence of signal transmission. The electroencephalographic signals are recorded through six channels, namely channel_1, channel_2, channel_3, channel_4, channel_5, and channel_6. Temperature signals are recorded via the channel_7, cerebral oxygen signals via the channel_8, and pressure signals via the channel_9. The maximum value of the six-channel pressure signals is displayed on the host computer. The upper left corner of the host computer software interface displays the status of the subject being measured, categorized into three states: safe, warning, and danger. When the pressure exerted by the doctor's handheld device on the surrounding cerebral tissue is excessive, the warning state will be displayed, alerting the doctor to reduce the pressure applied to the surrounding cerebral tissue. Additionally, all raw data is stored on the host computer and may be reviewed through the data review window. FIG. 6 exemplifies an electroencephalogram monitoring instance using a flexible hybrid electronic system for intraoperative intracranial monitoring. The left side displays the signal graphs of the six electroencephalographic channels, while the upper left corner of the right side shows a graph of frequency changes over time. The bottom right corner allows for the selection of power options, signal playback settings, and the number of channels for the time-frequency graph.

The corresponding usage platforms for the mobile terminal APP 12 are smartphones or PCs. The mobile terminal APP 12 is capable of real-time display of the measured subject's cerebral tissue oxygen saturation, pressure, temperature, and electroencephalographic signals. When the measured subject's pressure exceeds the predetermined normal pressure, the alarm function embedded within the APP will be activated. Surgeons performing operations may utilize the human-computer interaction APP on the mobile terminal to control the level of pressure applied to the cerebral tissue by the surgical instruments. The mobile terminal interaction APP may store the received cerebral tissue oxygen saturation, temperature, pressure, and electroencephalographic signals in a time-series format locally on the mobile terminal platform.

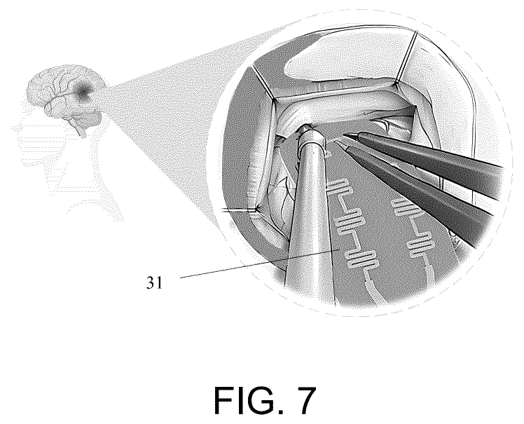

As illustrated in FIG. 7, the method of using a flexible hybrid electronic system for intraoperative intracranial monitoring is depicted. During the surgical procedure, this structure is gently inserted into the cranium using surgical tools such as forceps. Once properly positioned, the surgeon operates on the flexible front-end signal collection circuit 31. This approach not only reduces the pressure exerted by surgical instruments on the surrounding cerebral tissue but also enables real-time monitoring of intraoperative cerebral tissue oxygen levels, electroencephalographic signals, pressure signals, and temperature signals.

The disclosure, by utilizing printed circuit board technology, facilitates large-scale mass production. The disclosure employs a printed circuit board with a flexible PDMS substrate, covered with hydrogel adhesion on the surface, ensuring that the device may remain conformally attached to the subject's skin for extended periods, offering excellent wearing comfort. The system's flexible front-end signal collection circuit is designed with an array of a plurality of sensor units capable of real-time detection of parameters such as electroencephalogram, pressure, temperature, and cerebral tissue oxygen. Sensors for intraoperative intracranial monitoring must possess high sensitivity, a wide linear range, and stable data acquisition capabilities. The back-end signal processing circuit is modularly designed and composed of the plurality of collection board cards, allowing for modular combination of the collected parameters. The front-end sensor detection circuit and the back-end signal processing circuit are connected via FPC. The collection circuit transmits data to the hardware board cards, which, through a modular design, send the raw data to the upper system using Bluetooth, Wi-Fi, and serial communication for analysis, providing real-time display of the current state of cerebral tissue. The upper software design employs a Python and MATLAB interface for display. Although the success rate of craniotomies has exceeded 90% in recent years, the fragility of cerebral tissue necessitates utmost protection during surgery. Due to the complexity and difficulty of the surgical process, continuous monitoring of the patient's physiological state is essential. Typically, surgeons use cottoniod patties to protect the cerebral tissue, which may obstruct the corresponding monitoring areas. The present disclosure allows for the quantitative display of cerebral tissue oxygen, pressure, temperature, and electroencephalogram during surgery. When data anomalies are detected, a mobile terminal APP issues an alert and synchronizes the data to inform the surgical doctor, who may then take appropriate measures to address the situation. The present disclosure not only serves to protect cerebral tissue during craniotomy but also provides continuous monitoring of the patient's physiological state during surgery, issuing alerts when necessary, thereby contributing to the increased success rate of craniotomies and holding significant importance.

The aforementioned description constitutes merely the preferred embodiment of the present disclosure and is not intended to limit the disclosure. Any modifications, equivalent substitutions, improvements, and the like, made within the spirit and principles of the present disclosure, shall be encompassed within the scope to be protected by the present disclosure.

What is claimed is:

1. A flexible hybrid electronic system for intraoperative intracranial monitoring, comprising a flexible front-end signal collection circuit, a back-end signal processing circuit, and a host computer system;
wherein the flexible front-end signal collection circuit comprises a flexible framework body formed by a top encapsulation layer, an intermediate encapsulation layer, and a bottom substrate layer arranged from top to bottom, an electroencephalographic layer arranged between the intermediate encapsulation layer and the top encapsulation layer, a flexible sensor array disposed on an upper surface of the bottom substrate layer, a temperature-controlled hydrogel interface layer integrated above the top encapsulation layer, and an electrothermal film integrated below the bottom substrate layer, the electroencephalographic layer comprises a top substrate layer and an electroencephalographic electrode array and an internal circuit disposed on the top substrate layer, the electroencephalographic electrode array passes through the flexible framework body and connects with the temperature-controlled hydrogel interface layer, the flexible sensor array connects with the electroencephalographic layer through the intermediate encapsulation layer, the flexible sensor array is provided to detect a pressure signal, a temperature signal, and a cerebral tissue oxygen saturation signal, the electrothermal film controls adhesion of the temperature-controlled hydrogel interface layer through temperature, achieving adhesion and peeling of the temperature-controlled hydrogel interface layer with a cerebral tissue;
the back-end signal processing circuit is electrically connected with the flexible front-end signal collection circuit to receive the pressure signal, the temperature signal, an electroencephalographic signal, and the cerebral tissue oxygen saturation signal, analyse and process the signals, and then transmit the signals to the host computer system through wireless or wired transmission method.

2. The flexible hybrid electronic system for intraoperative intracranial monitoring according to claim 1, wherein the flexible sensor array comprises a pressure sensor array, a temperature sensor array, and a cerebral tissue oxygen sensor, which are respectively electrically connected with the back-end signal processing circuit.

3. The flexible hybrid electronic system for intraoperative intracranial monitoring according to claim 2, wherein a plurality of pressure sensors in the pressure sensor array are all flexible pressure sensors.

4. The flexible hybrid electronic system for intraoperative intracranial monitoring according to claim 2, wherein the back-end signal processing circuit comprises a collection circuit, a core processing circuit, a multiplexer switch, a flexible battery, a power management chip, and a wireless transmitter, the flexible battery achieves power supply to the wireless flexible hybrid electronic system through the power management chip, the flexible sensor array and the electroencephalographic electrode array are respectively electrically connected with the collection circuit, the collection circuit is connected to the core processing circuit through the multiplexer switch, the collection circuit is configured to receive the pressure signal, the temperature signal, the electroencephalographic signal, and the cerebral tissue oxygen saturation signal and transmit the signals to the core processing circuit through the multiplexer switch, the core processing circuit is configured to process the signals transmitted by the collection circuit and send the signals to the host computer system.

5. The flexible hybrid electronic system for intraoperative intracranial monitoring according to claim 4, wherein the collection circuit comprises an operational amplifier, an electroencephalogram collection front-end, and a cerebral oxygen collection front-end, the operational amplifier is electrically connected with the pressure sensor array and the temperature sensor array, the electroencephalogram collection front-end is electrically connected with the electroencephalographic electrode array, the cerebral oxygen collection front-end is electrically connected with the cerebral tissue oxygen sensor.

6. The flexible hybrid electronic system for intraoperative intracranial monitoring according to claim 4, wherein the core processing circuit transmits the processed signals to the host computer system through the wireless transmission module or through a serial port.

7. The flexible hybrid electronic system for intraoperative intracranial monitoring according to claim 1, wherein materials of the top encapsulation layer, the intermediate encapsulation layer, the bottom substrate layer, and the top substrate layer in the flexible front-end signal collection circuit are all polydimethylsiloxane, the electroencephalographic electrode array, the internal circuit, and wires connecting various components of the wireless flexible hybrid electronic system are all prepared from flexible composite conductive materials.

8. The flexible hybrid electronic system for intraoperative intracranial monitoring according to claim 1, wherein the temperature-controlled hydrogel interface layer comprises a plurality of temperature-controlled hydrogel patches corresponding to electroencephalographic electrodes one by one in the electroencephalographic electrode array, the plurality of temperature-controlled hydrogel patches respectively cover the corresponding electroencephalographic electrodes, the temperature-controlled hydrogel patches are formed through photo-initiated polymerization using poly(ethylene glycol) diacrylate as an initiator and poly(3,4-ethylenedioxythiophene) and poly(styrene acid) as blending components.

9. The flexible hybrid electronic system for intraoperative intracranial monitoring according to claim 8, wherein a thermally-induced actuator is disposed in the electrothermal film, heating sites of the thermally-induced actuator correspond to positions of the temperature-controlled hydrogel patches one by one.

10. The flexible hybrid electronic system for intraoperative intracranial monitoring according to claim 1, wherein the host computer system is provided with an alarm module, when pressure data received in the host computer system is higher than a preset normal pressure, the alarm module activates an alarm.
